(19) United States
(12) Patent Application Publication
(10) Pub. No.: US 2011/0200584 A1
(43) Pub. Date: Aug. 18, 2011

(54) METHOD TO STIMULATE IMMUNE FUNCTION AND REGENERATION

(75) Inventors: Frank Jakob Theodor Staal, Rotterdam (NL); Petrus Martinus Van Hagen, Rotterdam (NL); Aart Johannes Van Der Lelij, Rottedam (NL)

(73) Assignee: ERASMUS UNIVERSITY MEDICAL CENTER ROTTERDAM, Rotterdam (NL)

(21) Appl. No.: 12/996,367

(22) PCT Filed: Jun. 5, 2008
(86) PCT No.: PCT/NL2008/050354

§ 371 Date: May 4, 2011

(51) Int. Cl. A61K 39/395; A61K 38/22; C12N 5/078; A61P 37/04

(52) U.S. Cl. 424/130.1; 514/9.7; 435/377

(57) ABSTRACT

The present relates to a method to stimulate thymocytes to differentiate, proliferate and increase thymic output by administration of a hormone-based reagent.

### FIELD OF THE INVENTION

This invention relates to the field of immune stimulatory peptide hormones and derivatives and analogs thereof to improve or dampen the function of the adaptive immune system. More specifically, the invention relates to assessing the role of hormones, peptide derivates of such hormones, antibodies mimicking the function of such hormones or antagonizing their function, as well as synthetic agonists and antagonists of such hormones for stimulation of thymic output and increase of T cell reconstitution, the numbers of naïve T cells and TCR repertoire. The present invention relates inter alia to a method to stimulate immune function and T lymphocyte reconstitution in vivo

### BACKGROUND OF THE INVENTION

### T-Lymphocyte Reconstitution

Impaired T cell recovery following hematopoietic stem cell transplantation (HSCT) is currently considered to be the most important determinant of impaired immunocompetence in the late time period after HSCT. In addition, T cell numbers and functions are severely reduced after certain primary immunodeficiencies such as SCID, but importantly also after acquired immunodeficiencies such as those underlying HIV infection and other chronic viral infections. Especially, CD4⁺ T cell lymphocytopenia is associated with opportunistic infections. Also immune disbalances between effector and regulatory T cells may underlie many autoimmune diseases, in which a restored T cell repertoire may ameliorate autoimmune phenomena. Therefore, strategies to improve T cell recovery are expected to reduce treatment-related mortality and morbidity associated with HSCT, improve immune function in autoimmunity and restore T cell repertoire and function in primary and acquired autoimmune diseases and after intense chemotherapy of a wide variety of cancers. T cell recovery may occur either through thymic-dependent differentiation of bone marrow-derived progenitors cells into mature, naive T cells (thymopoiesis) or by homeostatic peripheral expansion (HPE) of mature peripheral T cells infused with the graft or residual host T cells. Thymopoiesis is considered important to generate a diverse TCR. However, thymopoiesis is severely hampered in adult stem cell graft recipients and adult patients, due to epithelial injury by chemo/radiotherapy, age-associated thymic involution, and graft-vs-host disease.

Several molecules, including IL2, IL-7, and SCF have been studied extensively in experimental bone marrow transplantation (BMT) models as possible thymopoietic and T cell restorative agents. IL-7 and IL-2 improve T cell recovery predominantly by peripheral expansion, whereas it marginally affects thymopoiesis, thereby not significantly increasing repertoire, naïve T cell numbers and thymic output. SCF encounters side effects on immature myeloid cells and HSCs.

Moreover, early acting cytokines such as stem cell factor and Fms-like tyrosine kinase 3 (Flt3) ligand (FL) are being explored as possible thymopoietic and T cell restorative agents. In addition, FL may enhance dendritic cell (DC)-driven homeostatic T cell expansion. FL indeed has some effects on the thymus, but in many conditions also stimulates other cells types, such as DCs and myeloid cells that indirectly stimulate T cell development in the periphery through peripheral expansion. Therefore, thus far no agents, growth factors or cytokines are described that specifically act on immature thymocytes to expand thymic output and TCR repertoire.

### Human T Cell Development

To gain more insight in human T cell development, Staal and coworkers. investigated gene expression profiles of thymocyte subsets using Affymetrix DNA microarrays to look for differences in gene expression during consecutive stages of T cell development.¹ Interestingly it was found that expression of certain hormone receptors were unregulated in subsets of thymocytes. One of these receptors, the TSH receptor (TSH-R), was upregulated in the DN stage, and down-regulated again in the DP stage (FIG. 2). These data suggest that TSH might play a role during T cell development.

T cell development occurs in the thymus, a primary lymphoid organ in which bone-marrow derived T cell precursors undergo a highly complex process of differentiation into mature T lymphocytes. T cell precursors, in the thymus called thymocytes, become committed to the T cell lineage and undergo proliferative expansion. Moreover a broadly diverse repertoire of T cell receptors (TCRs) is formed due to recombinant rearrangements of the Variable (V), Diversity (D) and Joiner (J) genes. Productive rearrangements result in TCR expression on the cell membrane. Thymocytes unable to express an functional TCR on their cell membrane will die by apoptosis, while thymocytes with a functional TCR will undergo positive and negative selection. During selection, thymocytes with too low or too high affinity for MHC molecules will die. Positively selected thymocytes will than differentiate into mature T lymphocytes.

The various stages of T cell differentiation can be recognized by expression of membrane bound co-receptors (FIG. 1). T cell precursors entering the thymus are called double negative (DN) referring to the absence of CD4 and CD8. In the DN stage there is enormous proliferative expansion and thymocytes become restricted to the T cell lineage. DN thymocytes become immature single positive thymocytes (ISP) recognized by CD4 positivity. During the DN and ISP stages the TCR is generated. In the next stage, the double positive stage (DP stage) recognized by CD4 and CD8 positivity, thymocytes that possess a working T cell receptor will undergo positive and negative selection. Selected thymocytes differentiate into mature single positive (SP) lymphocytes, recognized by positivity for CD4 or CD8.

Differentiation of thymocytes is under control of many factors including cytokines, adhesion molecules, hormones and transcription factors. However a more detailed understanding of T cell development is necessary.

### Thyroid Stimulating Hormone (TSH) and TSH Receptor (TSH-R)

Thyroid stimulating hormone (TSH) is a hormone operating within the hypothalamus-pituitary-thyroid axis in which it regulates endocrine function of the thyroid gland. TSH synthesis in the anterior pituitary is stimulated by thyrotropin-releasing hormone (TRH) released from the hypothalamus and inhibited by thyroid hormones in a negative-feedback loop. Physiological roles of TSH in the thyroid gland include iodine uptake and organification, production and release of thyroid hormones, and promotion of thyroid growth. Moreover TSH plays an important role in ontogeny of the thyroid gland.

TSH is a 28- to 30-kDA glycoprotein of the glycoprotein hormone family which also includes FSH, LH and hCG. They all consist of a common α-subunit and a unique β-subunit. The genes encoding the α- and β-subunit of TSH are located on chromosome 6 and 1 respectively. The α- and β-subunit consist of 92 and 118 amino acids respectively.

TSH binds the G protein-coupled TSH-R which leads to a second messenger pathway involving predominantly cAMP, IP3 and DAG, ultimately resulting in modulation of gene expression.

TSH-R is a member of the G protein coupled receptor (GPCR) superfamily. The extracellular domain contains 300 to 400 amino acids. The gene for the TSH-R is on chromosome 14q31.

TSH is predominantly located on the follicular epithelial cells of the thyroid. However TSH-R is also expressed in several other tissues including adipocytes, retro-ocular fibroblasts, neuronal cells and astrocytes (mRNA transcripts and/or protein). Moreover TSH-R has been found on lymphocytes and recently TSH-R has been found in the thymus as well.

### TSH and the Immune System

It has long been known that TSH-R is expressed on hematopoietic cells. Flow cytometry and Western blot analysis demonstrated expression of TSH-R on immune cells from human PBMC (peripheral blood mononuclear cells). TSH-R is mainly expressed on monocytes/macrophages and NK-cells, with no or very low expression on B and T cells.^{2, 3}

Moreover TSH-R was demonstrated in the human thymus by immunohistochemistry. Here TSH-R was present in Hassall' corpuscles and their surrounding epithelial cells.⁴ More recently TSH-R was found by PCR analysis in the thymus in patients with Graves disease. The nucleotide sequence was equal to TSH-R in the thyroid gland.^{4, 5}

Although TSH-R is found on immune cells and in the thymus, not much is known about the function of TSH in the immune system. In rat thymus it was possible to stimulate TSH-R with 1 mU/ml TSH, measured by cAMP production, showing that the TSH-R is a functional receptor in the thymus.⁶ In hypothyroid rats involution of the spleen, lymph nodes and thymus occurs, while hyperthyroidism causes an increase in size of lymph nodes. However TSR-R knockout mice do not seem to have major immunologic abnormalities, although no studies were performed with this question.⁷ In humans a few studies have been performed to investigate immunologic changes in hyper- and hypothyroidism. These studies show an increase in CD4/CD8-ratio in hyperthyroidism (Graves' disease) and a decrease in CD4/CD8-ratio in hypothyroidism (Hashimoto thyroiditis).^{8, 9} However it is thought that these changes might be involved in the pathogenesis of hyper/hypothyroidism instead of being a consequence of changes in TSH levels. Moreover in hyper- and hypothyroidism not only TSH but also T3 and T4 levels change and T3 and T4 have been shown to interact with the immune system as well.¹⁰

### SUMMARY OF THE INVENTION

The present invention provides in a first aspect a method to stimulate thymocytes to differentiate, proliferate and/or increase thymic output by administration to said thymocytes of a hormone-based reagent.

The present invention also provides a method to stimulate lymphocyte function by administration of a hormone-based reagent to a mammal in need of such stimulation or to stimulate thymocytes to differentiate, proliferate and/or increase thymic output by administration of said reagent in vivo.

The methods of the invention can be used in vitro and/or in vivo.

In in vitro methods, the thymocytes are exposed to the hormone based reagent in for instance a tissue culture. In in vivo methods, the hormone based reagent may be administered to the mammal in which the thymocyte differentiation, proliferation or thymic output is to be stimulated or increased. The hormone-based reagent can be administered orally, nasally or parenterally, intraveneously (iv), intramuscularly (im) or locally, mucosal or dermal.

The thymocytes used in aspects of the present invention can in principle be thymocytes from any mammal. Preferably, the thymocytes are from a human, a rat, a mouse, or a non human primate. The mammal in aspects of the invention may be a non-human mammal, but is preferably a human.

By the term “thymic output” is well known in the art and can be determined by analysis of T-cell-receptor gene rearrangement excisional circles or T cell receptor excision circles (TREC). (see reference 1).

A method that can be used in vitro and in vivo to stimulate lymphocyte function by administration (orally, nasal or parenteral, iv, im or locally mucosal or dermal) of a hormone-based reagent.

In a method of the invention said administration of a hormone based reagent may involve the administration of a peptide hormone from the pituitary gland, stomach or placenta.

The hormone based reagent may suitably be thyroid-stimulating hormone (TSH) or a TSH-receptor (TSH-R) agonist, such as an antibody, a small molecule pharmacological peptide and/or a non-peptide agonist.

A method of the invention may further involve the step wherein another substance is administered in order to stimulate the (expression of the) TSH-R or one of its subunits.

In a method of the present invention the hormone based reagent may suitably be luteinizing hormone, human chorionic gonadotropin or related hormones or peptide or non-peptide analogs thereof.

In a method of the present invention the hormone based reagent may suitably be an antibody or a fragment thereof. Preferably the fragment exhibits a binding that is functionally equivalent to the binding of the complete antibody.

A method of the present invention may be used to improve at least T cell and immune function and or repertoire after bone marrow transplantation or hematopoietic stem cell transplantation.

In particular, the method of the present invention may be used to improve T cell numbers and or function and or repertoire during or after HIV disease.

Alternatively, or additionally, a method of the present invention may be used to improve T cell numbers and or function and or repertoire after chemotherapy for haematological malignancies and solid tumors.

Alternatively, or additionally, a method of the present invention may be used to improve T cell numbers and or function and or repertoire after immunodeficiencies of known or unknown origin.

Alternatively, or additionally, a method of the present invention may be used to improve T cell numbers and or function and or repertoire in elderly.

A method of the present invention may be performed or used in model organisms such as non human primates, mice or rats.

A method of the present invention may be performed or used in clinical protocols in combination with other drugs to enhance immune function.

The invention further relates to compositions, such as pharmaceutical compositions, comprising a hormone based reagent as defined herein for use in a method of the present invention.

### BRIEF DESCRIPTION OF THE DRAWINGS

FIG. 1 shows normal T cell development.

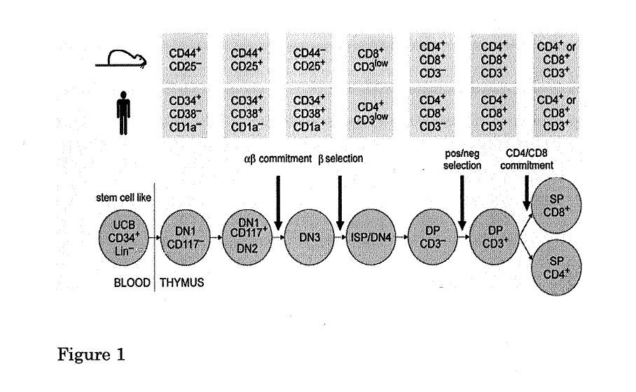

FIG. 2 shows TSHr expression in thymocytes.

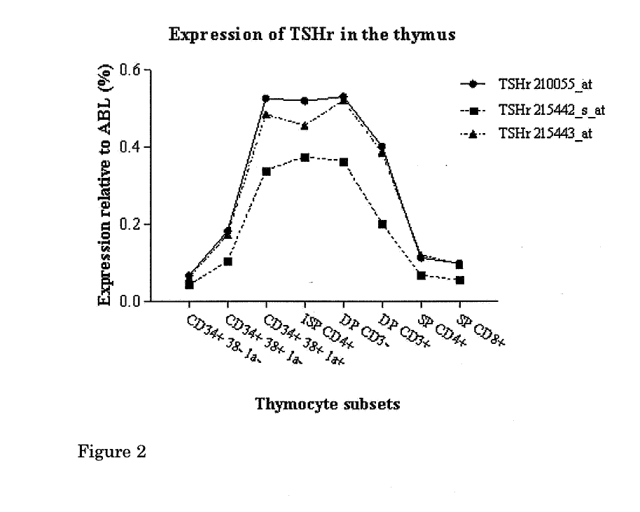

FIG. 3 shows the effect of addition of TSH to the culture system resulted in an increase of ISP and DP thymocytes in 6 out of 7 cultures. A: control, B: 0.1 nM TSH.

### DESCRIPTION OF THE PREFERRED EMBODIMENTS

The present invention relates to the field of immune stimulatory peptide hormones and derivatives and analogs thereof to improve or dampen the function of the immune system. The invention provides a method for stimulation of thymic output and increase of T cell reconstitution and TCR repertoire. It is based on findings relating to TSH and TSH-R but may relate to other hormones binding this or related receptors. It can be used in humans and model organisms in which TSH-R or related receptors perform such thymic or immune cell function. Application areas are wide and include but are not limited to immune reconstitution after BMT and HSCT, in immunodeficiencies, in autoimmune diseases and after chemotherapy.

From the prior art studies it is clear that TSH-R is present in the thymus and therefore TSH could play a role in T cell development. TSH does not seem to play a major role in physiological situations demonstrated by the lack of spontaneous immunologic problems in TSH-R knockout mice and in hyper/hypothyroidism. However TSH-R in the thymus might be a potential novel therapeutic target in lymphopenic patients.

To further investigate the role of TSH in T cell development we performed experiments in an advanced culture system, a fetal thymic organ culture (FTOC), in which human stem cells are cultured in mouse fetal thymic tissue. Cells were cultured for 14 days with or without addition of 1 nM TSH bi-weekly. The effects of TSH on differentiation, apoptosis/cell death and proliferation were investigated. After 14 days of culture stem cells were fully T cell committed with clear differentiation into ISP and DP thymocytes. Preliminary data show that addition of TSH to the culture system resulted in an increase in ISP and DP thymocytes in 6 out of 7 cultures. Moreover cell death was clearly reduced in cultures with TSH. Proliferation was not highly affected, however thymocytes are already highly proliferating cell which makes it hard to show small differences in proliferation.

Moreover to learn more about the mechanism in which TSH influences T cell development RT-PCR analysis was performed on thymocytes and thymic epithelial cells. TSH-R was demonstrated on thymocytes as well as on thymic epithelial cells, however TSH was not present in the thymus. So TSH might directly stimulate thymocytes or stimulate epithelial cells which in turn can stimulate thymocytes.

In literature TSH-R is also found on monocytes and mature T and B cells using biotinylated TSH and flow cytometry. However microarray data present in our lab of mature B and T cells and monocytes show no expression of TSH-R in those cells, nor do flow cytometric analyses using TSH-R specific antibodies Microarray analyses indicate at least a 100 fold higher expression on proliferating thymocyte subsets, minimal expression on resting thymocytes and no expression on peripheral T cells Theoretically these compounds would therefore only stimulate thymic T cell production, not peripheral, homeostatic expansion.

Concluding, concentrations of 0.1-10 nM TSH stimulate differentiation and decrease cell death of human thymocytes in vitro demonstrating that the TSH-R is functional in human thymus enabling TSH to act as a previously unrecognized growth factor for human thymocytes. Therefore TSH might be a novel therapeutic modality in all kind of patients with lymphocyte depletion for example following cytoreductive therapies or in disease states such as HIV.

### TSH in Other Non-Endocrine Organs

### Bone Metabolism in Thyroid Disease

Hyperthyroidism is associated with osteoporosis whereas hypothyroidism is associated with high bone mass and increased cortical width.¹¹ These bone abnormalities were thought to be the result of abnormal thyroid hormone levels and not of abnormal TSH levels. However recently in the literature conflicting results were found about the role of TSH in bone metabolism. Abe et al. demonstrated that TSHR null mice developed severe osteoporosis. Substitution of thyroid hormones failed to reverse this effect, indicating a role for TSH in the pathogenesis of osteoporosis.¹² However Bassett et al. demonstrated that a hypothyroid mouse model with normal TSH-R and a hypothyroid mouse model with no functional TSH-R both developed reduced cortical bone deposition and reduced mineralisation. This indicates a role for thyroid hormones rather than TSH in bone metabolism.¹³

Due to the inverse relationship between TSH and thyroid hormones it is difficult to determine which hormones are responsible for the effects on bone metabolism. However since the TSH-R is present on osteoclasts as well as on osteoblasts, TSH itself might influence bone metabolism.

### Lipid Metabolism in Thyroid Disease

Hypothyroidism is associated with increases in concentrations of total cholesterol, LDL cholesterol and often HDL cholesterol. In hyperthyroidism total cholesterol and LDL cholesterol are decreased, and HDL is normal or low.

It is known that thyroid hormones stimulate de novo cholesterol synthesis in the liver, this leads to a decreased synthesis of cholesterol in hypothyroidism and increased synthesis in hyperthyroidism. However thyroid hormones are also able to regulate LDL receptor expression on liver cells. Therefore high levels of thyroid hormones in hyperthyroidism increase turnover of LDL cholesterol leading to lower levels of LDL cholesterol. In hypothyroidism turnover is decreased leading to high LDL levels.¹⁴ So thyroid hormones play a role in dyslipidemia in thyroid diseases, the role of TSH is highly unknown.

### Other Hormones Capable of Binding to TSH-R

It should be noted that LH and HCG can also bind to the TSH-R and may similarly stimulate TSH-R mediated thymic output. Therefore—in analogy with the invention described above for TSH, TSH-R agonizing antibodies and agonists—similar effects on thymic output and T cell reconstitution could be expected with above mentioned other compounds, hormones or derivates thereof.

The invention provides a method for stimulation of thymic output and increase T cell reconstitution and TCR repertoire. It is based on findings relating to TSH and TSH-R, but may relate to other hormones binding TSH-R or related receptors. To exemplify the invention, we will describe a clinical protocol using rhTSH.

### EXAMPLES

### Study Design

A single center, double blind randomised, clinical trial.

### Study Population

50 patients treated for hypothyroidism, with stable disease over the past six months.

### Study Treatments

All subjects will receive rhTSH (Thyrogen®) derived from genzyme Europe BV (The Netherlands, Naarden) in a dose of 0.9 mg two times a week intramuscular for four weeks.

Recombinant Human TSH

rhTSH is a highly purified synthetic human thyroid stimulating hormone produced in a genetically modified Chinese hamster ovary cell line.

Since 2001 the use of rhTSH is approved by the European Agency for the Evaluation of Medicinal Products for the diagnostic monitoring of patients with differentiated thyroid cancer. Standard treatment of differentiated thyroid cancer includes thyroidectomy and chemotherapy. Follow-up includes thyroglobulin testing both on TSH suppression therapy and after TSH stimulation. TSH stimulation was performed by withdrawal of thyroid hormone. However the induction of hypothyroidism often reduces patient's quality of life. Treatment with rhTSH makes it possible to raise TSH levels without inducing hypothyroidism.¹⁶

### Dose

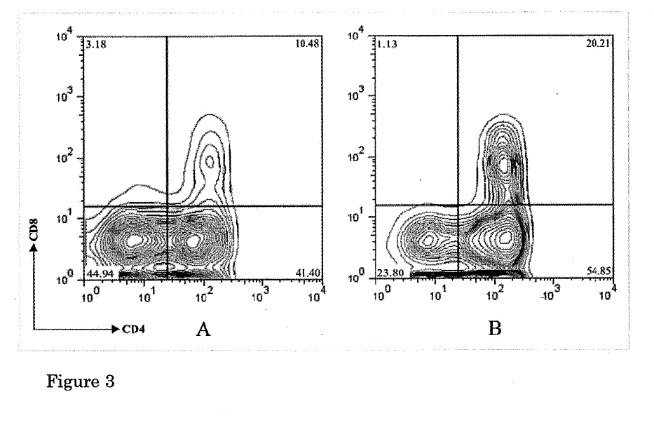

In the follow up of differentiated thyroid cancer patients receive two gifts of rhTSH 0.9 mg i.m. once a day for two consecutive days. Studies performed with this medication scheme showed a rapid elevation of TSH levels with maximal levels 4 to 8 hours after injection of rhTSH. Genzyme Nederland collected data from studies in which 2 doses of 0.9 mg were given in patients with thyroid cancer. Based on these data a time curve for TSH levels over time for administration of 0.9 mg once a day for two consecutive days was constructed using mathematical models (FIG. 3). Moreover some studies are performed in healthy subjects to evaluate the response to a single dose of different concentrations of rhTSH i.m. These data are summarized in FIG. 4.¹⁷⁻¹⁹ Half life time of a single dose of 0.9 mg rhTSH i.m. was reported to be 25±10 hours in patients with thyroid cancer.²⁰

So rhTSH can be given safely in a dose of 0.9 mg i.m. once a day for two consecutive days. However in this study it will be necessary to give rhTSH for a prolonged period of time to affect T cell development. In this study patients will receive 0.9 mg rhTSH i.m. twice a week for 4 weeks. Based on above data on doses of rhTSH and height of TSH levels this will lead to prolonged elevated levels of TSH without the risk of accumulation

### Primary Endpoints

Primary endpoint consist of a change in cell numbers or ratio's of peripheral T cell subpopulations in response to treatment with rhTSH. T cell subpopulations will be defined using flow cytometry. Moreover thymic output will be measured using TREC analysis.

### Secondary Endpoints

Secondary endpoints are:

Thymus volume, measured by CT

Cardial function, measured by blood pressure and ECG

Lipid metabolism, measured by TG, LDL, HDL, total cholesterol

Bone metabolism, measured by alkaline phosphatase

CK levels

### REFERENCES

1. Dik W A, Pike-Overzet K, Weerkamp F, et al. New insights on human T cell development by quantitative T cell receptor gene rearrangement studies and gene expression profiling. J Exp Med 2005; 201(11):1715-23.
2. Coutelier J P, Kehrl J H, Bellur S S, Kohn L D, Notkins A L, Prabhakar B S. Binding and functional effects of thyroid stimulating hormone on human immune cells. J Clin Immunol 1990; 10(4):204-10.
3. Chabaud O, Lissitzky S. Thyrotropin-specific binding to human peripheral blood monocytes and polymorphonuclear leukocytes. Mol Cell Endocrinol 1977; 7(1):79-87.
4. Murakami M, Hosoi Y, Negishi T, et al. Thymic hyperplasia in patients with Graves' disease. Identification of thyrotropin receptors in human thymus. J Clin Invest 1996; 98(10):2228-34.
5. Nakamura T, Murakami M, Horiguchi H, et al. A case of thymic enlargement in hyperthyroidism in a young woman. Thyroid 2004; 14(4):307-10.
6. Murakami M, Hosoi Y, Araki O, et al. Expression of thyrotropin receptors in rat thymus. Life Sci 2001; 68(25):2781-7.
7. Marians R C, Ng L, Blair H C, Unger P, Graves P N, Davies T F. Defining thyrotropin-dependent and -independent steps of thyroid hormone synthesis by using thyrotropin receptor-null mice. Proc Natl Acad Sci USA 2002; 99(24):15776-81.
8. Bossowski A, Urban M, Stasiak-Barmuta A. Analysis of changes in the percentage of B (CD19) and T (CD3) lymphocytes, subsets CD4, CD8 and their memory (CD45RO), and naive (CD45RA) T cells in children with immune and non-immune thyroid diseases. J Pediatr Endocrinol Metab 2003; 16(1):63-70,
9. Covas M I, Esquerda A, Garcia-Rico A, Mahy N. Peripheral blood T-lymphocyte subsets in autoimmune thyroid disease. J Investig Allergol Clin Immunol 1992; 2(3):131-5.
10. Villa-Verde DM, de Mello-Coelho V, Farias-de-Oliveira D A, Dardenne M, Savino W. Pleiotropic influence of triiodothyronine on thymus physiology. Endocrinology 1993; 133(2):867-75.
11. Sun L, Davies T F, Blair H C, Abe E, Zaidi M. TSH and bone loss. Ann N Y Acad Sci 2006; 1068:309-18.
12. Abe E, Marians R C, Yu W, et al. TSH is a negative regulator of skeletal remodeling. Cell 2003; 115(2):151-62.
13. Bassett J H, Williams A J, Murphy E, et al. A lack of thyroid hormones rather than excess TSH causes abnormal skeletal development in hypothyroidism. Mol Endocrinol 2007.
14. Duntas L H. Thyroid disease and lipids. Thyroid 2002; 12(4):287-93.
15. LeMar H J, Jr., West S G, Garrett C R, Hofeldt F D. Covert hypothyroidism presenting as a cardiovascular event. Am J Med 1991; 91(5):549-52.
16. Sugino K, Ito K, Takami H. Management of differentiated thyroid carcinoma with radioiodine and recombinant human TSH. Endocr J 2006; 53(6):723-8.
17. Ramirez L, Braverman L E, White B, Emerson C H. Recombinant human thyrotropin is a potent stimulator of thyroid function in normal subjects. J Clin Endocrinol Metab 1997; 82(9):2836-9.
18. Torres M S, Ramirez L, Simkin P H, Braverman L E, Emerson C H. Effect of various doses of recombinant human thyrotropin on the thyroid radioactive iodine uptake and serum levels of thyroid hormones and thyroglobulin in normal subjects. J Clin Endocrinol Metab 2001; 86(4):1660-4.
19. Nielsen V E, Bonnema S J, Hegedus L. Effects of 0.9 mg recombinant human thyrotropin on thyroid size and function in normal subjects: a randomized, double-blind, cross-over trial. J Clin Endocrinol Metab 2004; 89(5):2242-7.
20. Emerson C H, Torres M S. Recombinant human thyroid-stimulating hormone: pharmacology, clinical applications and potential uses. BioDrugs 2003; 17(1):19-38.
21. Luster M. Acta Oncologica Lecture. Present status of the use of recombinant human TSH in thyroid cancer management. Acta Oncol 2006; 45(8):1018-30.
22. Genzyme Europe BY T N, Naarden. Patient medication guide. 2007.

1-14. (canceled)

15. A method to stimulate thymocyte differentiation in a mammalian subject comprising administering to a subject in need thereof an effective amount of a hormone-based reagent, wherein said hormone-based reagent is thyroid-stimulating hormone (TSH) or a TSH-R agonist.

16. The method according to claim 15, wherein said hormone-based reagent is TSH.

17. The method according to claim 15, wherein said TSH-R agonist is an antibody or antibody fragment.

18. The method according to claim 15, wherein said TSH-R agonist is luteinizing hormone, human chorionic gonadotropin, or a peptide or non-peptide analog thereof.

19. The method according to claim 15, wherein said TSH-R agonist is a small molecule pharmacological peptide.

20. The method according to claim 15, wherein said subject is in need of improving at least T cell and immune function and/or repertoire after bone marrow transplantation or hematopoietic stem cell transplantation.

21. The method according to claim 15, wherein said subject is in need of improving T cell numbers and/or function and/or repertoire during or after HIV disease

22. The method according to claim 15, wherein said subject is in need of improving T cell numbers and/or function and/or repertoire after chemotherapy for haematological malignancies and solid tumors

23. The method according to claim 15, wherein said subject is in need of improving T cell numbers and/or function and/or repertoire after immunodeficiencies of known or unknown origin.

24. The method according to claim 15, wherein said subject is elderly.

25. The method according to claim 15, wherein said subject is a laboratory model organism.

26. The method according to claim 15, wherein said subject is enrolled in a clinical protocol and said reagent is administered in combination with other drugs to enhance immune function.

27. The method according to claim 15, wherein said reagent is administered orally, nasally or parenterally, intravenously (iv), intramuscularly (im) or locally, mucosal or dermal.

28. An in vitro method to stimulate thymocytes to differentiate comprising administering a hormone-based reagent to thymocytes, wherein said hormone-based reagent is thyroid-stimulating hormone (TSH) or a TSH-R agonist.
